(19) United States
(12) Patent Application Publication
(10) Pub. No.: US 2018/0085460 A1
(43) Pub. Date: Mar. 29, 2018

(54) METHODS OF TREATING PROSTATITIS

(71) Applicant: Aquinox Pharmaceuticals (Canada) Inc., Vancouver (CA)

(72) Inventors: Lloyd F. Mackenzie, North Vancouver (CA); Jennifer Cross, Langley (CA); Curtis Harwig, Vancouver (CA)

(21) Appl. No.: 15/716,322

(22) Filed: Sep. 26, 2017

Related U.S. Application Data
(60) Provisional application No. 62/399,894, filed on Sep. 26, 2016.

(51) Int. Cl. A61K 45/00; C07C 211/60

(52) U.S. Cl. CPC A61K 45/00; C07C 211/60

(57) ABSTRACT

This invention is directed to the use of a SHIP1 activator, preferably AQX-1125, to treat prostatitis.

### FIELD OF THE INVENTION

This invention is directed to the use of a SHIP1 activator to treat prostatitis.

### BACKGROUND OF THE INVENTION

Prostatitis is often described as swelling and inflammation of the prostate gland. It can occur in the presence or absence of detectable infection. Just 5% to 10% of cases are caused by bacterial infection.

Prostatitis can affect men of all ages. According to the National Institutes of Health, prostatitis may account for up to 25% of all office visits for complaints involving the genital and urinary systems of young and middle-aged men. In fact, chronic prostatitis is the number-one reason men under age 50 visit a urologist. In some cases, chronic prostatitis follows an attack of acute prostatitis. Chronic prostatitis may also be related to other urinary tract infections. Prostatitis is considered chronic if it lasts more than three months.

Types of prostatitis include:

A. Acute Bacterial Prostatitis.

Acute bacterial prostatitis is a sudden bacterial infection marked by inflammation of the prostate. This is the least common form of prostatitis, but the symptoms are usually severe. Patients with this condition have an acute urinary tract infection with increased urinary frequency and urgency, a need to urinate a lot at night, and have pain in the pelvis and genital area. They often have fever, chills, nausea, vomiting, and burning when urinating. Acute bacterial prostatitis requires prompt treatment, as the condition can lead to bladder infections, abscesses in the prostate or, in extreme cases, completely blocked urine flow. Left untreated, the condition can cause confusion and low blood pressure, and may be fatal. The condition is usually treated in the hospital with intravenous antibiotics, pain relievers, and fluids.

B. Chronic Bacterial Prostatitis.

The primary symptom of chronic infectious prostatitis is usually repeated bladder infections. This condition is the result of recurrent urinary tract infections that have entered the prostate gland. It is thought to exist for several years in some men before producing symptoms. The symptoms are similar to acute bacterial prostatitis, but are less severe and can fluctuate in intensity. The diagnosis of this condition is often challenging. It is often difficult to find the bacteria in the urine. Treatment includes antibiotics for four to 12 weeks and other treatment for pain. Sometimes men are given suppressive low-dose, long-duration antibiotic therapy.

C. Chronic Prostatitis/Chronic Pelvic Pain Syndrome (CP/CPPS).

This is the most common form of prostatitis, accounting for 90% of the cases. The condition is marked by urinary and genital pain for at least three of the past six months. Patients have no detectable bacteria in their urine, but may have other signs of inflammation. The condition can be confused with interstitial cystitis (a chronic inflammation of the bladder).

D. Asymptomatic Inflammatory Prostatitis.

This is characterized by inflammation of the prostate without genitourinary tract symptoms.

Pharmacological activation of SHIP1 has emerged as a novel approach to regulate inflammation for the following reasons:
1. The PI3K/SHIP1 pathway (see FIG. 1) is an established target for drug development.
2. The PI3K/SHIP1 pathway plays a key role in regulating cell migration and activation.
3. Targeting SHIP1 is an alternate way of modulating the PI3K pathway.
4. SHIP1 expression is restricted to hematopoietic derived cells which limits off-target toxicity.
5. SHIP1 activation redirects cellular PI3K signaling, rather than preventing it.

AQX-1125 is the first clinical-stage, once-daily, orally administered, SHIP1 activator with demonstrated anti-inflammatory effects in animals and humans (see Stenton et al., 2013; Br. J. Pharmacol., 168: 1519-29, Leaker et al., 2014; Clin. Exp. Allergy., 44: 1146-53 and Nickel et al., 2016, The Journal of Urology, Vol. 196, No. 3, pp. 747-754.

AQX-1125, which is disclosed and claimed in U.S. Pat. No. 7,601,874, is the acetate salt of (1S,3S,4R)-4-((4R,7aS)-4-(aminomethyl)-7a-methyl-1-methyleneoctahydro-1H-inden-5-yl)-3-(hydroxymethyl)-4-methylcyclohexanol. Positive effects of AQX-1125 have been previously observed in several preclinical models of inflammation and bladder pain syndrome.

Accordingly, there exists a need for a SHIP1 activator in the treatment of prostatitis.

### SUMMARY OF THE INVENTION

This invention is directed to the use of a SHIP1 activator in treating prostatitis in a mammal.

Accordingly, in one aspect, this invention is directed to a method of treating prostatitis in a mammal, wherein the method comprises administering a therapeutically effective amount of an SHIP1 activator, preferably AQX-1125, to the mammal in need thereof.

In another aspect, this invention is direct to a method of treating prostatitis in a mammal, wherein the method comprises administering a therapeutically effective amount of a pharmaceutical composition comprising a pharmaceutically acceptable excipient and a therapeutically effective amount of SHIP1 activator, preferably AQX-1125, to the mammal in need thereof.

In another aspect, this invention is directed to the use of a SHIP1 activator, preferably AQX-1125, for the treatment of prostatitis in a mammal.

In another aspect, this invention is directed to the use of a SHIP1 activator, preferably AQX-1125, in the preparation of a medicament for the treatment of prostatitis.

In another aspect, this invention is directed to the use of a composition comprising a SHIP1 activator, preferably AQX-1125, and an excipient for the treatment of prostatitis in a mammal.

### BRIEF DESCRIPTION OF THE DRAWINGS

FIG. 1 illustrates the PI3K/SHIP1 pathway.

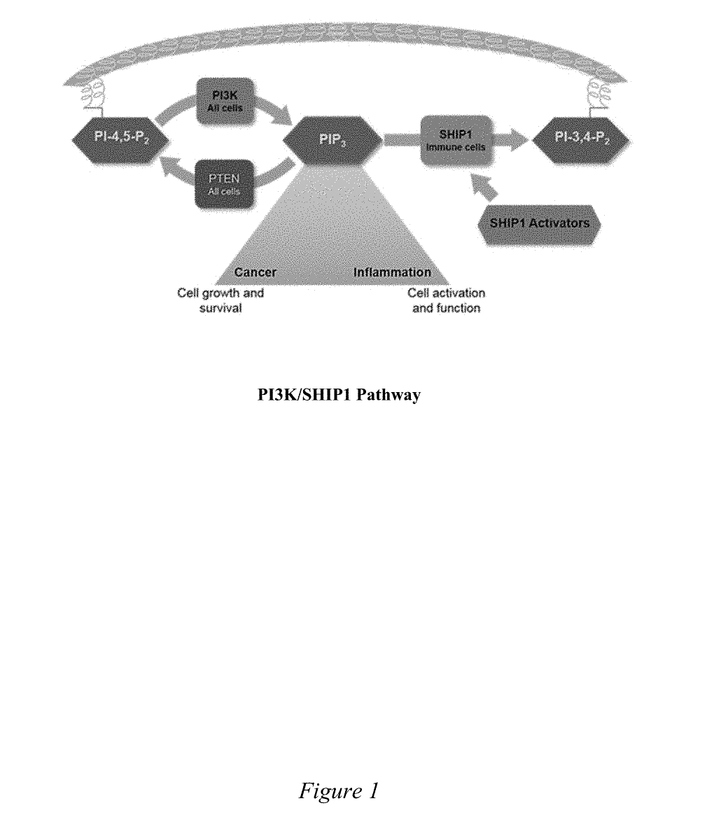

FIG. 2 illustrates the design of the study described herein.

FIG. 3 illustrates nociceptive thresholds before (i.e., basal value) and 1, 3 and 7 days after saline or carrageenan i.prost. injection in vehicle-treated rats. Results in FIG. 3 are expressed as mean±s.e.m. (n=10/group). ns p>0.05, *** p<0.001, Mann-Whitney test.

FIG. 4 illustrates nociceptive thresholds before (i.e., basal value) and 1, 3 and 7 days after saline or carrageenan i.prost. injection in vehicle-treated rats. Results in FIG. 4 are expressed as mean±s.e.m. (n=10/group). ns p>0.05, *** p<0.001, two-way RM ANOVA.

FIG. 5 illustrates AUC (0.16-2 g) before (i.e., basal value) and 1, 3 and 7 days after saline or carrageenan i.prost. injection in vehicle-treated rats. Results in FIG. 5 are expressed as mean±s.e.m. (n=10/group). ns p>0.05, *** p<0.001, unpaired t test or Mann Whitney.

FIG. 6 illustrates ventral prostate lobes weight expressed either in mg (A) or as a ratio of the whole prostate weight (B), 7 days after saline or carrageenan i.prost. injection in vehicle-treated rats. Results in FIG. 6 are expressed as mean±s.e.m. (n=10/group). ns p>0.05, unpaired t test.

FIG. 7 illustrates nociceptive thresholds (A), nociceptive scores (B) and AUC (0.16-2 g) (C), before (i.e., basal value) and 3 days after carrageenan i.prost. injection in vehicle- and ibuprofen-treated groups. Results in FIG. 7 are expressed as mean±s.e.m. (n=10/group). ns p>0.05, *** p<0.001, Mann-Whitney test, two-way RM ANOVA or Unpaired t test.

FIG. 8 illustrates nociceptive thresholds before (i.e., basal value) and 1, 3 or 7 days after carrageenan i.prost. injection in vehicle- and AQX-1125 (0.3, 1, 3 or 10 mg/kg)-treated groups. Results are expressed in FIG. 8 as mean±s.e.m. (n=10/group). ns p>0.05, one-way ANOVA between the groups and ns p>0.05, * p<0.05, ** p<0.01, *** p<0.001 Mann-Whitney test v. Vehicle.

FIG. 9 illustrates nociceptive scores before (i.e., basal value) and 1, 3 or 7 days after carrageenan i.prost. injection in vehicle- and AQX-1125 (0.3, 1, 3 or 10 mg/kg)-treated groups. Results are expressed in FIG. 9 as mean±s.e.m. (n=10/group). ns p<0.05, two-way RM ANOVA between the groups and p=0.0638, *p<0.05, **p<0.01, *** p<0.001, two-way RM ANOVA vs Vehicle.

FIG. 10 illustrates AUC (0.16-2 g) (i.e., basal value) and 1, 3 or 7 days after carrageenan i.prost. injection in vehicle- and AQX-1125 (0.3, 1, 3 or 10 mg/kg)-treated groups. In FIG. 9, ns p>0.05, one way ANOVA between the groups and ns p>0.05, * p<0.05, ** p<0.01, *** p<0.001 Mann-Whitney test or Unpaired t test w/or w/o Welch's correction vs Vehicle.

FIG. 11 illustrates ventral prostate lobes weight expressed either in mg (A) or as a ratio of the whole prostate weight (B), 7 days after carrageenan i.prost. injection in vehicle- and AQX-1125 (0.3, 1, 3 or 10 mg/kg)-treated groups. Results are expressed in FIG. 11 as mean±s.e.m. (n=10/group). ns p>0.05, Mann-Whitney or Unpaired t test vs Vehicle.

### DETAILED DESCRIPTION OF THE INVENTION

As noted above in the Summary of the Invention, this invention is directed to methods of treating prostatitis in a mammal, wherein the method comprises administering a therapeutically effective amount of AQX-1125 or administering a therapeutically effective amount of a pharmaceutical composition comprising a pharmaceutically acceptable excipient and a therapeutically effective amount of AQX-1125 to the mammal in need thereof.

In particular, this invention is directed to methods of treating prostatitis in a mammal, wherein the method comprises administering a therapeutically effective amount of AQX-1125 or administering a therapeutically effective amount of a pharmaceutical composition comprising a pharmaceutically acceptable excipient and an effective amount of AQX-1125 to the mammal in need thereof, wherein the prostatitis is selected from acute bacterial prostatitis, chronic bacterial prostatitis, asymptomatic inflammatory prostatitis or chronic prostatitis/chronic pelvic pain syndrome (CP/CPPS)

The preparation of AQX-1125 and pharmaceutical compositions comprising same, is disclosed and claimed in U.S. Pat. Nos. 7,601,874 and 7,999,010, the relevant disclosures of which are incorporated by reference in their entireties.

Inflammatory chronic prostatitis/chronic pelvic pain syndrome (CP/CPPS), also referred as NIH category IIIA prostatitis, is characterized by pelvic pain due to nonbacterial prostate inflammation (Nickel, J C. et al., 2008. Category III Chronic Prostatitis/Chronic Pelvic Pain Syndrome: Insights from The National Institutes of Health Chronic Prostatitis Collaborative Research Network Studies. Curr. Urol. Rep. 9: 320-327). It is often associated with irritative and obstructive voiding symptoms including urgency, frequency and hesitancy (Krieger J N. et al., 1999. NIH consensus definition and classification of prostatitis. JAMA. 282: 236-237). Symptoms can also include sexual dysfunction and pelvic pain. Although CP/CPPS is the most common type of prostatitis and accounts for 90-95% of prostatitis diagnoses, it has no well-defined aetiology and limited treatment options (Habermacher G M. et al., 2006. Prostatitis/chronic pelvic pain syndrome. Annu. Rev. Med. 57: 195-206).

Considerable attention has been focused on creating reliable animal models of CP/CPPS (Vyskhovanets E V. et al., 2007. Experimental rodent models of prostatitis: limitations and potential. Prostate Cancer Prostatic Dis. 10: 15-29). However, only few of them have been evaluated for pain although it is the hallmark symptom of CP/CPPS.

An animal model of inflammation-induced pelvic pain for inflammatory CP/CPPS was developed and validated using intraprostatic (i.prost.) injection of carrageenan in male Sprague-Dawley rats (adapted from Radhakrishnan, R. and Nallu, R. S., 2009. Development and characterization of a novel animal model of prostate inflammation-induced chronic pelvic pain, Inflammopharmacology, 17: 23-28). This animal model was characterized by both referred mechanical allodynia (decreased mechanical threshold in response to innocuous von Frey forces) and referred mechanical hyperalgesia (increased nociceptive scores in response to noxious von Frey forces) (Augé C. et al., 2012, Nociceptive measures in chronic prostatitis: a model of inflammation-induced pelvic pain in rats, 14th World Congress on Pain (Milan, Italy)).

A previous study showed that repeated oral treatments (once a day during 11 days) of AQX-1125 (3, 10 and 30 mg/kg) had strong anti-nociceptive properties on carrageenan-induced pelvic pain in this animal model. Maximal effect of AQX-1125 was reached at the dose of 3 mg/kg.

AQX-1125 effects were evaluated on pelvic pain response at 0.3, 1, 3 and 10 mg/kg doses (oral administration) in the carrageenan-induced prostatitis model in male Sprague-Dawley rats. Ibuprofen was used in the study as a reference substance.

### Definitions

As used in the specification and appended claims, unless specified to the contrary, the following terms have the meaning indicated:

“Prostatitis” includes acute bacterial prostatitis, chronic bacterial prostatitis, chronic prostatitis/chronic pelvic pain syndrome (CP/CPPS) and asymptomatic inflammatory prostatitis.

“Mammal” includes humans and both domestic animals such as laboratory animals and household pets, (e.g., cats, dogs, swine, cattle, sheep, goats, horses, rabbits), and non-domestic animals such as wildlife and the like. Preferably the mammal is a human.

“Therapeutically effective amount” refers to that amount of a compound of the invention or that amount of a pharmaceutical composition of the invention which, when administered to a mammal, preferably a human, is sufficient to effect treatment, as defined below, of prostatitis in the mammal, preferably a human. The amount of a compound of the invention or the amount of the pharmaceutical composition of the invention which constitutes a “therapeutically effective amount” will vary depending on the compound, the composition, the condition and its severity, the manner of administration, and the age of the mammal to be treated, but can be determined routinely by one of ordinary skill in the art having regard to his own knowledge and to this disclosure.

“Treating” or “treatment” as used herein covers the treatment of prostatitis in a mammal, preferably a human, having prostatitis, and includes:

(a) preventing prostatitis from occurring in a mammal, in particular, when such mammal is predisposed to prostatitis but has not yet been diagnosed as having it;

(b) inhibiting prostatitis, i.e., arresting its development;

(c) relieving (or ameliorating) prostatitis, i.e., causing regression of prostatitis; or

(d) relieving (or ameliorating) the symptoms resulting from prostatitis, e.g., relieving pain without addressing the underlying cause.

AQX-1125 refers to the compound which is the acetate salt of (1S,3S,4R)-4-((4R,7aS)-4-(aminomethyl)-7a-methyl-1-methyleneoctahydro-1H-inden-5-yl)-3-(hydroxymethyl)-4-methylcyclohexanol, and has the following chemical structure:

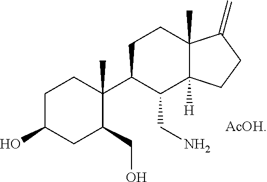 [SMILES: C=C1CC[C@H]2[C@H](CN)[C@@H]([C@@]3(C)CC[C@H](O)C[C@@H]3CO)CC[C@]12C]

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

A “pharmaceutical composition” refers to a formulation of a compound of the invention and a medium generally accepted in the art for the delivery of the biologically active compound to mammals, e.g., humans. Such a medium includes all pharmaceutically acceptable excipients therefor.

### Abbreviations

The following abbreviations are used herein:

% represents Percent.
° C. represents Degree Celsius.
ANOVA represents Analysis of Variance.
AUC represents Area Under the Curve.
CP/CPPS represents Chronic Prostatitis/Chronic Pelvic Pain Syndrome.
D represents Day.
g represents Gram.
h represents Hour.
i.p. represents Intraperitoneal.
i.prost. represents Intraprostatic.
kg represents Kilogram.
mg represents Milligram.
min represents Minute.
mL represents Milliliter.
NIH represents National Institute of Health.
ns represents Not Significant.
p.o. represents Per os (orally).
RM represents Repeated Measure.
s.e.m. represents Standard Error of the Mean.
μm represents Micrometer.
w/v represents Weight/Volume.

### Embodiments of the Invention

As noted above in the Summary of the Invention, one aspect of the invention is a method of treating prostatitis in a mammal, wherein the method comprises administering a therapeutically effective amount of an SHIP1 activator to the mammal in need thereof.

Another aspect of the invention is a method of treating prostatitis in a mammal, wherein the method comprises administering a therapeutically effective amount of a pharmaceutical composition comprising a pharmaceutically acceptable excipient and a therapeutically effective amount of SHIP1 activator to the mammal in need thereof.

Of the two aspects of the invention noted above, certain embodiments are preferred.

One embodiment is wherein the SHIP1 activator is AQX-1125.

Another embodiment is wherein the prostatitis is selected from acute bacterial prostatitis, chronic bacterial prostatitis, chronic prostatitis/chronic pelvic pain syndrome (CP/CPPS) and asymptomatic inflammatory prostatitis.

Another embodiment is wherein the prostatitis is acute bacterial prostatitis.

Another embodiment is wherein the prostatitis is chronic bacterial prostatitis.

Another embodiment is wherein the prostatitis is asymptomatic inflammatory prostatitis.

Another embodiment is wherein the prostatitis is chronic prostatitis/chronic pelvic pain syndrome (CP/CPPS).

Another embodiment is wherein the therapeutically effective amount of the SHIP1 activator, preferably AQX-1125, administered to the mammal is from about 0.1 mg/kg to about 5.0 mg/kg.

Another embodiment is wherein the therapeutically effective amount of the SHIP1 activator, preferably AQX-1125, administered to the mammal is from about 0.2 mg/kg to about 4.0 mg/kg.

Another embodiment is wherein the therapeutically effective amount of the SHIP1 activator, preferably AQX-1125, administered to the mammal is from about 0.3 mg/kg to about 3.75 mg/kg.

Another embodiment is wherein the therapeutically effective amount of the SHIP1 activator, preferably AQX-1125, administered to the mammal is from about 2.0 mg/kg to about 3.5 mg/kg.

Another embodiment is wherein the therapeutically effective amount of the SHIP1 activator, preferably AQX-1125, is administered to the mammal once a day, twice a day, three times a day or four times a day.

Another embodiment is wherein the therapeutically effective amount of the SHIP1 activator, preferably AQX-1125, is administered to the mammal once a day.

Another embodiment is wherein the therapeutically effective amount of the SHIP1 activator, preferably AQX-1125, is orally administered to the mammal.

Of the above embodiments, one preferred embodiment is wherein the prostatitis is chronic prostatitis/chronic pelvic pain syndrome (CP/CPPS), wherein the therapeutically effective amount of AQX-1125 administered to the mammal is from about 2.0 mg/kg per day to about 3.5 mg/kg per day, wherein the therapeutically effective amount of AQX-1125 is administered to the mammal once a day and wherein the therapeutically effective amount of AQX-1125 is orally administered to the mammal.

Another aspect of the invention is the use of a SHIP1 activator for the treatment of prostatitis in a mammal.

Another aspect of the invention is the use of a SHIP1 activator in the preparation of a medicament for the treatment of prostatitis.

In one embodiment of the two aspects of the invention described above, the SHIP1 activator is AQX-1125.

In another embodiment of these two aspects, the prostatitis is selected from acute bacterial prostatitis, chronic bacterial prostatitis, chronic prostatitis/chronic pelvic pain syndrome (CP/CPPS) and asymptomatic inflammatory prostatitis. Preferably, the prostatitis is chronic prostatitis/chronic pelvic pain syndrome (CP/CPPS).

In another aspect, this invention is directed to the use of a composition comprising a SHIP1 activator, preferably AQX-1125, and an excipient for the treatment of prostatitis, preferably chronic prostatitis/chronic pelvic pain syndrome (CP/CPPS), in a mammal.

It is understood that any of the embodiment described above can be combined with one of more specific embodiments to define an embodiment not specifically disclosed herein.

Specific embodiments of the various aspects of the invention are described in more detail below.

### Study Methods and Materials

Adult male Sprague-Dawley rats (Janvier Labs, Le Genest Saint Isle, France), weighing 395±52 g at the beginning of the experiments, were used. Animals were acclimatized to the laboratory conditions for at least 3 days before the start of experiments. Rats were housed in groups of 2 in polysulfone type Sealsafe plus GR900 cages (Tecniplast, Lyon, France) on a bed of wood chips with free access to food and water ad libitum. Species appropriate environmental enrichment was added to the cages. The animal house was maintained under artificial lighting 7:00 a.m. to 7:00 p.m. in a controlled ambient temperature of 22±2° C., and relative humidity maintained at 55±10%.

Mechanical stimulation was performed using von Frey filaments of different forces.

The whole prostate and ventral prostate lobes were weighed using a Scout® Pro balance (OHAUS, Nänikon, Switzerland).

A stock solution of AQX-1125, at a final concentration of 2 mg/mL (w/v, free base form), was prepared once a week in physiological saline and stored at +4° C. For each day of administration, the stock solution was allowed to equilibrate to room temperature under stirring, for at least 30 min. The stock solution was used as it is for the 10 mg/kg dose group or it was diluted 3.33-, 10- or 33.33-fold in vehicle (physiological saline) to get final concentrations of 0.6 mg/mL, 0.2 mg/mL or 0.06 mg/mL for the 3 mg/kg, 1 mg/kg or 0.3 mg/kg dose group, respectively.

A stock solution of Ibuprofen ((RS)-2-(4-(2-methylpropyl)phenyl)propanoic acid, i.e. the reference compound) was prepared fresh on the day of administration at a final concentration of 60 mg/mL (w/v, free base form) in vehicle (physiological saline).

Carrageenan was purchased from Sigma-Aldrich. Carrageenan (prepared fresh each day of injection) was dissolved in sterile physiological saline at a final concentration of 30 mg/mL (w/v).

### Experimental Groups

Seven experimental groups of 10 animals were included in this study as described in the table below.

| Group | Treatment (p.o.) | Injection (i.prost.) | n |
|---|---|---|---|
| 1 | Vehicle (saline) | Saline | 10 |
| 2 | Vehicle (saline) | Carrageenan | 10 |
| 3 | AQX-1125 (0.3 mg/kg) | Carrageenan | 10 |
| 4 | AQX-1125 (1 mg/kg) | Carrageenan | 10 |
| 5 | AQX-1125 (3 mg/kg) | Carrageenan | 10 |
| 6 | AQX-1125 (10 mg/kg) | Carrageenan | 10 |
| 7 | Ibuprofen (300 mg/kg) | Carrageenan | 10 |

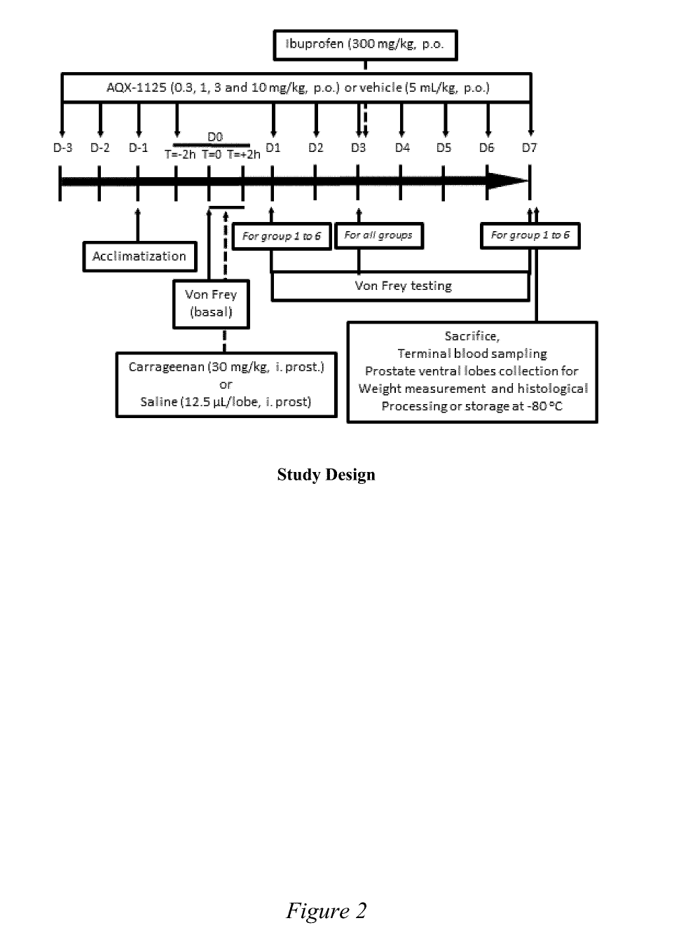

### Study Design (see FIG. 2)

AQX-1125 (0.3, 1, 3 and 10 mg/kg) or saline vehicle were administered orally (p.o.) once a day from D−3 to D+7 for a total of 11 administrations. At D0, D+1, D+3 and D+7 administrations were performed 2 h prior to von Frey testing. Ibuprofen was administered p.o. once at D+3, 1 h prior to von Frey testing. On the morning of each experimental day, rats were weighed and administrations were performed at a volume of 5 mL/kg.

Surgical procedures were performed in morning, as follows: anaesthesia was induced by 5% isoflurane and maintained with 2.5% isoflurane for the duration of the procedure. The surgical area (lower abdomen above the penis) and the scrotal skin were shaved. An incision was made in the skin and abdominal wall to expose both ventral lobes of the prostate gland. Carrageenan (30 mg/mL) was injected in both right and left ventral lobes of the prostate (˜12.5 μL per lobe).

Control rats were injected with physiological saline under the same experimental conditions as carrageenan (i.e. i.prost., ˜12.5 μL per lobe).

After i.prost. injection, the wounds in both muscles were closed using absorbable suture and the skin layers were closed using permanent suture.

### Assessment of Pelvic Pain Using Von Frey Filaments

Pelvic pain was evaluated in a non-blinded manner by applying 6 von Frey filaments of increasing forces (0.16, 0.4, 0.6, 1, 1.4 and 2 g) to the scrotum area, with an interval of 5 seconds between each filament.

Prior to testing, the scrotal skin area designated for mechanical stimulation of each animal was shaved. Animals were placed on a raised wire mesh floor under individual transparent Plexiglas boxes and acclimatized for at least 30 min before starting the von Frey test. Filaments were then applied 1-2 seconds through the mesh floor with enough strength to cause the filament to slightly bend. Each filament was tested 3 times with an interval of 5 seconds between each application.

Nociceptive behavioral scores to von Frey filaments were assessed just before saline or carrageenan injection, in order to obtain basal values (D0), and at defined time points (D+1, D+3 and D+7) after saline or carrageenan injection.

Nociceptive behaviors were scored for each animal and each filament as follows:

| Score | Behavior |
|---|---|
| 0 | no response |
| 1 | reaction of the animal (e.g. retraction of the testicle) |
| 2 | jumping |
| 3 | licking of the site stimulated with von Frey filaments |

Animals were anesthetized by intraperitoneal (i.p.) injection of pentobarbital (54.7 mg/mL, 0.5 mL/rat). Blood samples were collected under deep anesthesia through the abdominal vein by syringe aspiration and transferred into K3-EDTA coated-tubes. Blood samples were centrifuged (1 000 g for 10 min at +4° C.) and 1 mL of the supernatants were collected and stored at −20° C. for further analysis.

After blood sampling, rats were sacrificed by cervical dislocation and the whole prostates were rapidly collected and weighed. Then both ventral prostate lobes were dissected and weighed together.

Each of the ventral prostate lobes were cut in transverse section into two approximately equal pieces. A piece was defined as half of both right and left ventral prostate lobes. One piece was used for histological processing and one piece was stored at −80° C. for future analysis.

The samples for histological analysis were transferred into tissue processing embedding cassettes, fixed in 4% buffered formaldehyde and kept at room temperature for 24 h. Samples were then transferred into ethanol 70% and stored at +4° C. for potential histology.

### Results of Study

Carrageenan (30 mg/mL, i.Prost.) Induced Pelvic Pain Up to 7 Days Post-Injection:

In order to confirm carrageenan-induced pelvic pain up to 7 days post-injection, the nociceptive parameters between saline- and carrageenan-injected groups (in the presence of vehicle) were compared.

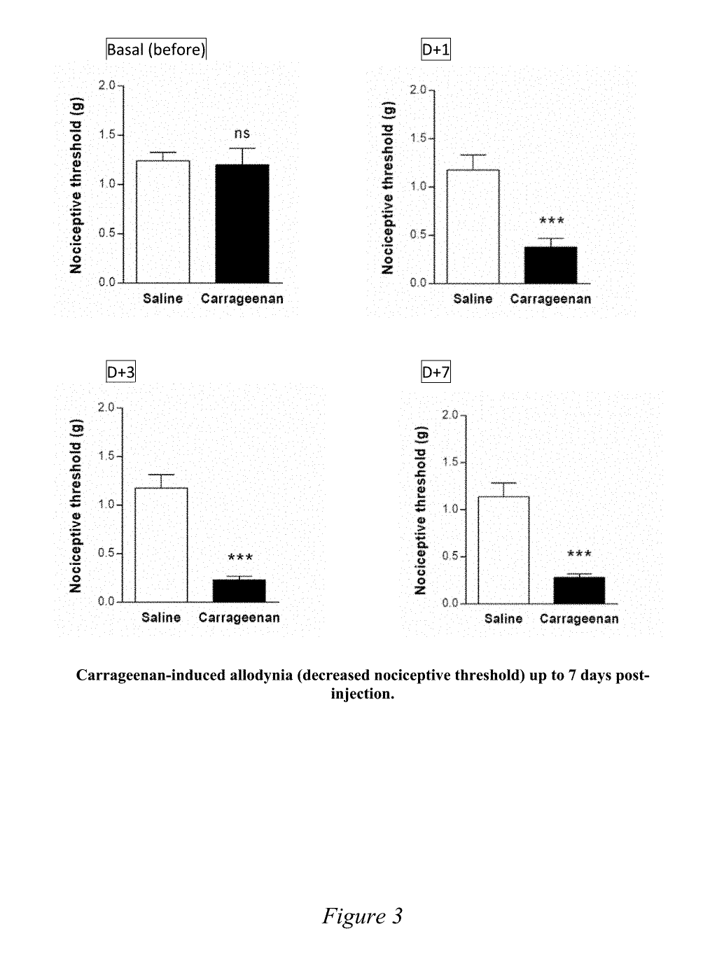

Before carrageenan injection (30 mg/mL, i.prost.) or saline injection (i.e., basal values), no significant difference in the nociceptive parameters was observed between both groups (p>0.05, FIGS. 3-5, “Basal”).

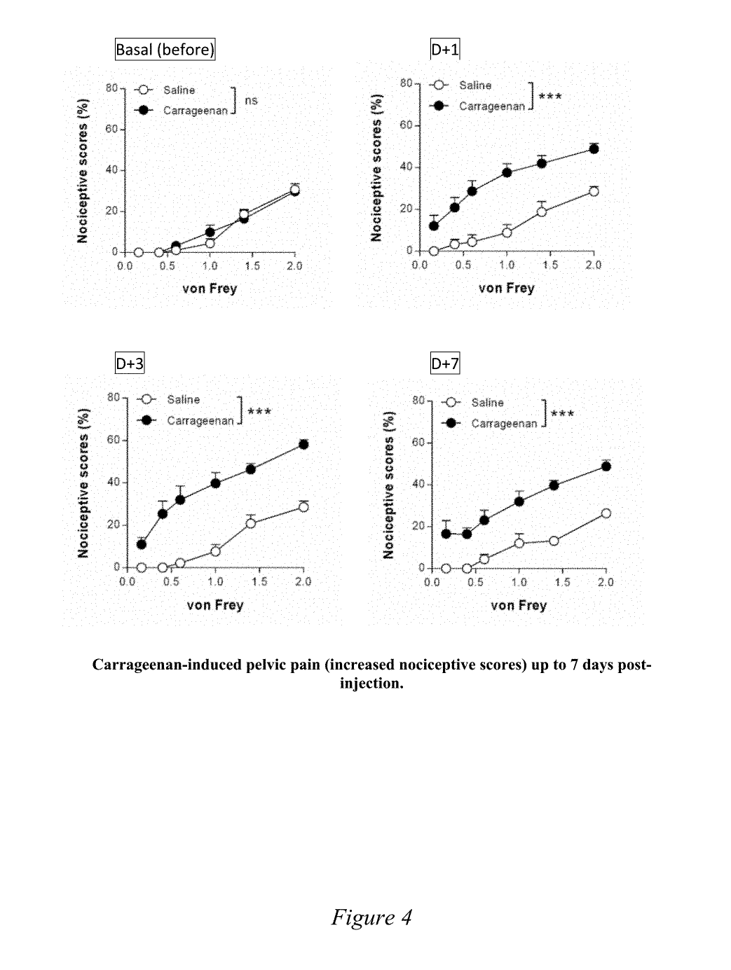

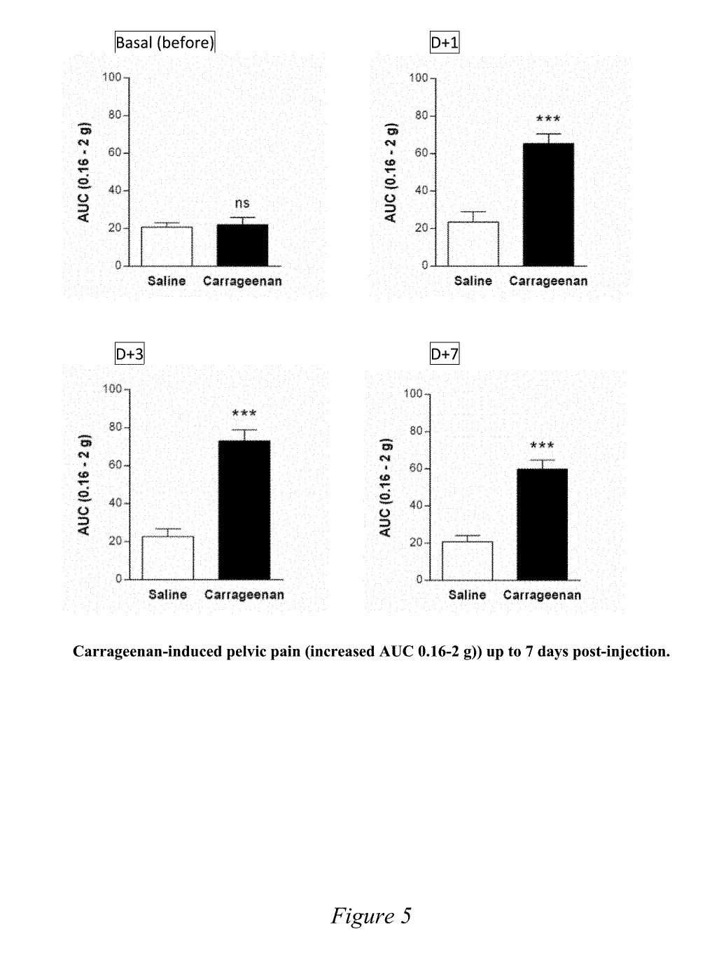

A significant decrease in the nociceptive threshold was observed 1, 3 and 7 days after carrageenan injection as compared to the saline injected group (p<0.001, FIG. 3). In addition, carrageenan increased the nociceptive scores (p<0.001, FIG. 4) and the corresponding AUC (0.16-2 g) (p<0.001, FIG. 5).

Effect of Carrageenan (30 mg/mL, i.Prost.) on Ventral Prostate Lobes Weight at D+7:

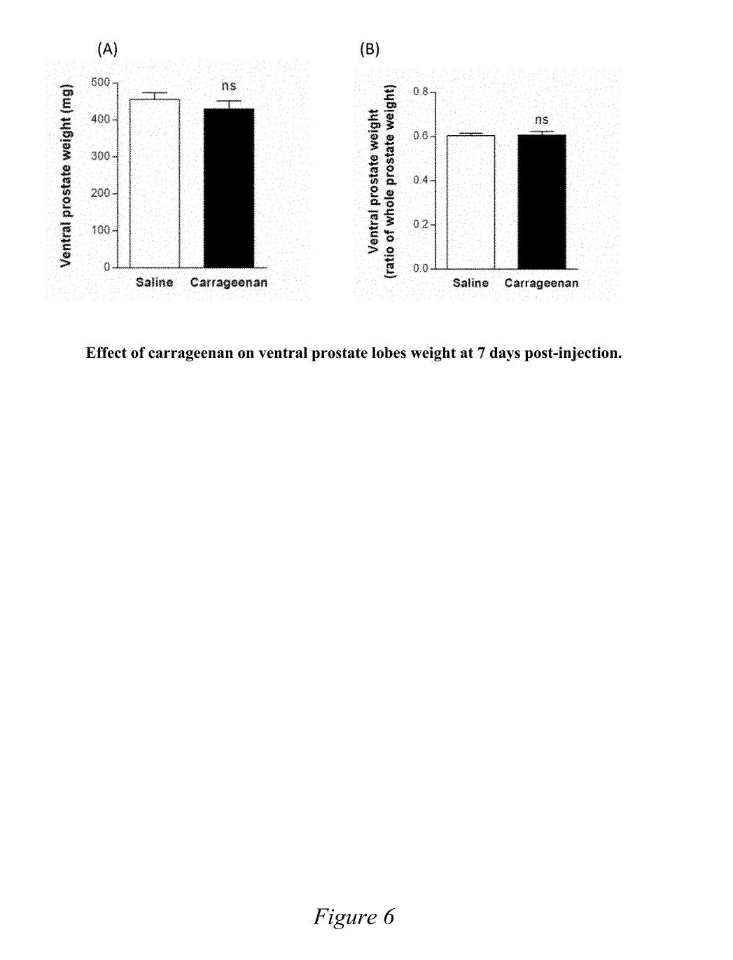

At day 7 post-carrageenan injection, no difference in the ventral prostate lobe weights, expressed either in mg or as a ratio of whole prostate weight, were observed as compared to saline (p>0.05, FIGS. 6(A) and 6(B), respectively).

Ibuprofen (300 mg/kg, p.o.) alleviated carrageenan-induced pelvic pain:

To validate the model, the nociceptive parameters between vehicle- and ibuprofen-treated (300 mg/kg, p.o.) groups were compared.

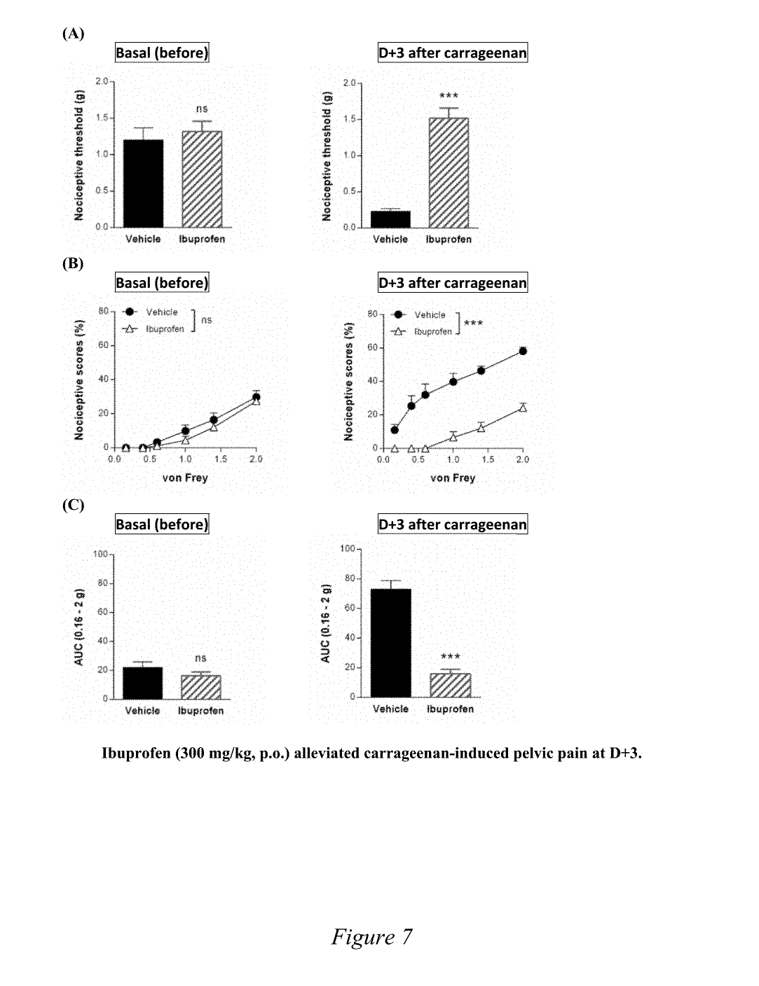

Before carrageenan injection, no significant difference in the nociceptive parameters was observed between both groups (p>0.05, FIGS. 7(A), 7(B), and 7(C), “Basal”).

Three days after carrageenan injection, a significant increase in the nociceptive threshold was observed after ibuprofen treatment as compared to vehicle (p<0.001, FIG. 7(A), “D+3 after carrageenan”). In addition, decreases in nociceptive scores and corresponding AUC (0.16-2 g) were observed in the Ibuprofen group (p<0.001, FIGS. 7(B) and 7(C), “D+3 after carrageenan”, respectively).

AQX-1125 (0.3, 1, 3 or 10 mg/kg, p.o.) Alleviated Carrageenan-Induced Pelvic Pain:

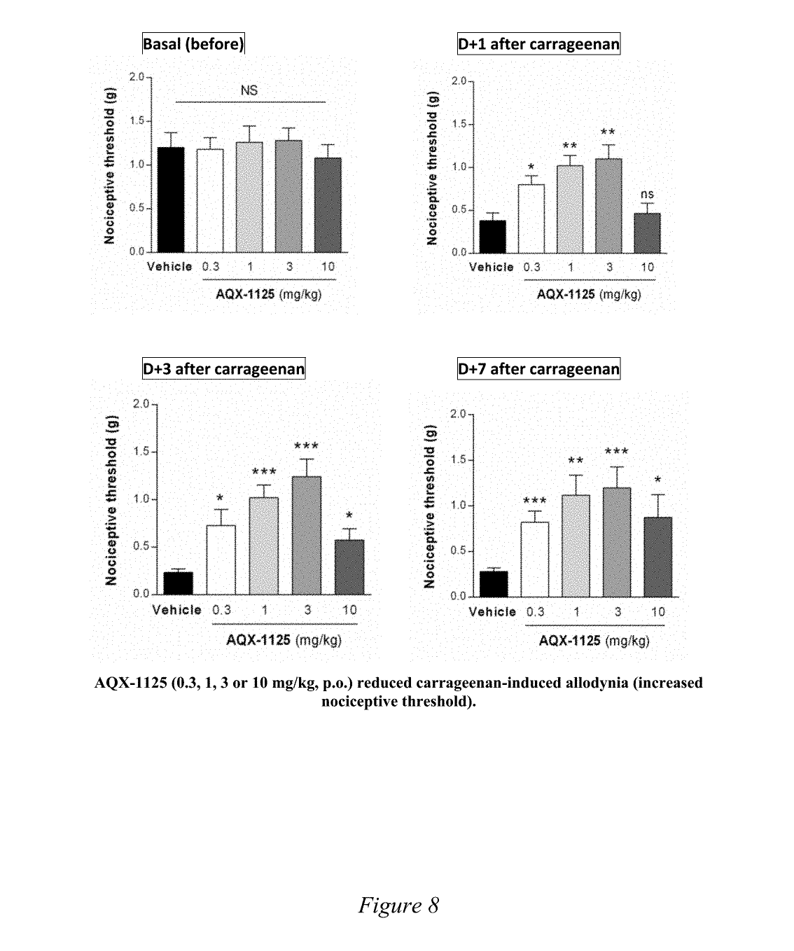

Before carrageenan injection (i.e., basal values), no significant difference in the nociceptive parameters was observed between groups (p>0.05, FIGS. 8-10, “Basal”).

In order to analyze the effects of AQX-1125 (0.3, 1, 3 or 10 mg/kg) oral administration on carrageenan-induced pelvic pain, nociceptive parameters were compared between the vehicle and the four AQX-1125-treated groups.

At day 1, 3 and 7 post-carrageenan injection, AQX-1125 dosed at 0.3, 1, and 3 mg/kg significantly and dose-dependently increased the nociceptive threshold, as compared to vehicle (p<0.05, FIG. 8). At 10 mg/kg, AQX-1125 effect reached the significance level only at day 3 and 7 (p<0.05 and p<0.05, FIG. 8, “D+1 after carrageenan” (p>0.05) and “D+3 and D+7 after carrageenan” (p<0.05), respectively).

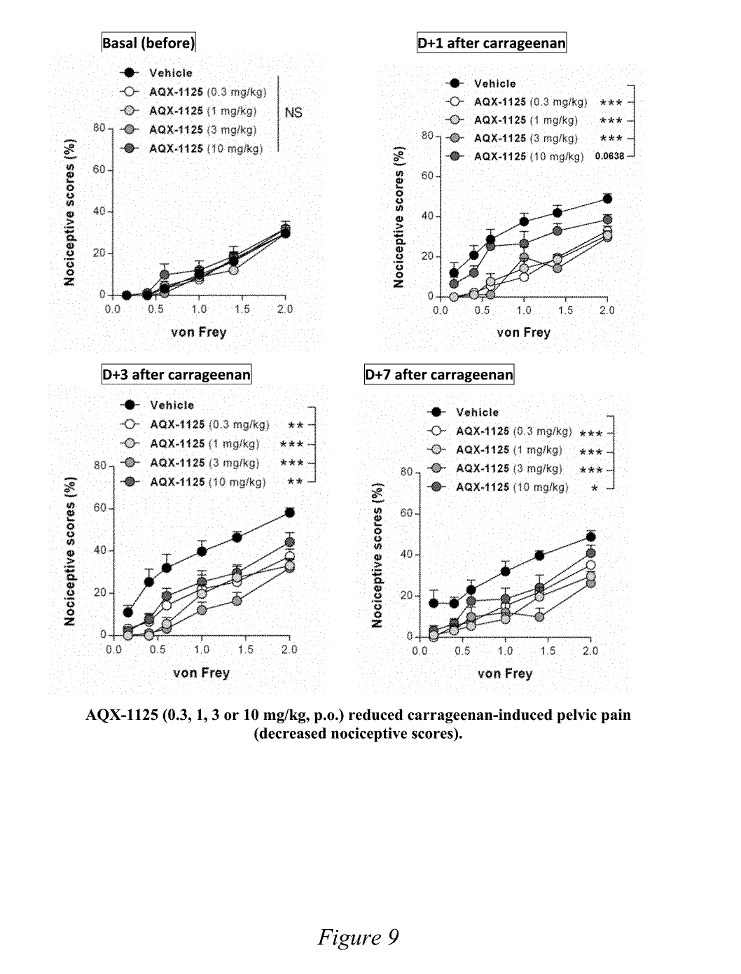

In addition, at all tested time-points, decreases in nociceptive scores were observed with AQX-1125 at all doses tested (FIG. 9). AQX-1125 effects reached significance, except for the 10 mg/kg dose at D1 (p=0.0638, FIG. 9, “D+1 after carrageenan”).

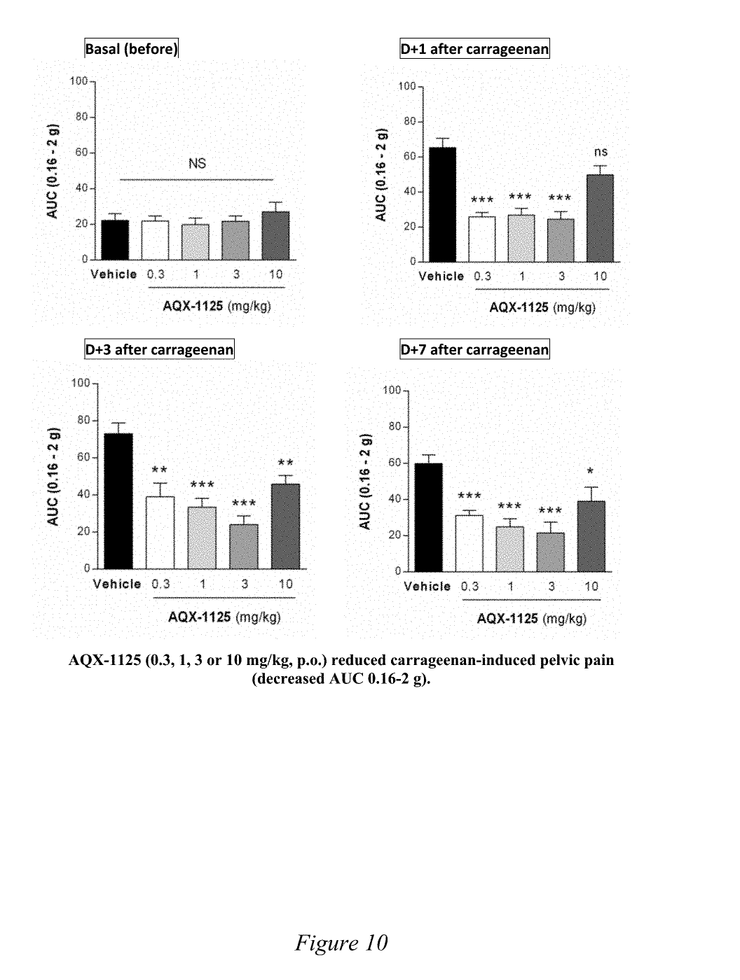

Similar results were obtained for corresponding AUC (0.16-2 g). At all tested time-points, significant decreases in AUC (0.16-2 g) were observed with the 0.3, 1, and 3 mg/kg doses of AQX-1125 (p<0.01, FIG. 10). At the 10 mg/kg dose, a significant effect was observed only at day 3 and 7 (p>0.05, p<0.01 and p<0.05, FIG. 10, “D+1 after carrageenan”, “D+3 after carrageenan” and “D+7 after carrageenan”, respectively).

Effect of AQX-1125 (0.2, 1, 3 or 10 mg/kg, p.o.) on Ventral Prostate Lobes Weight:

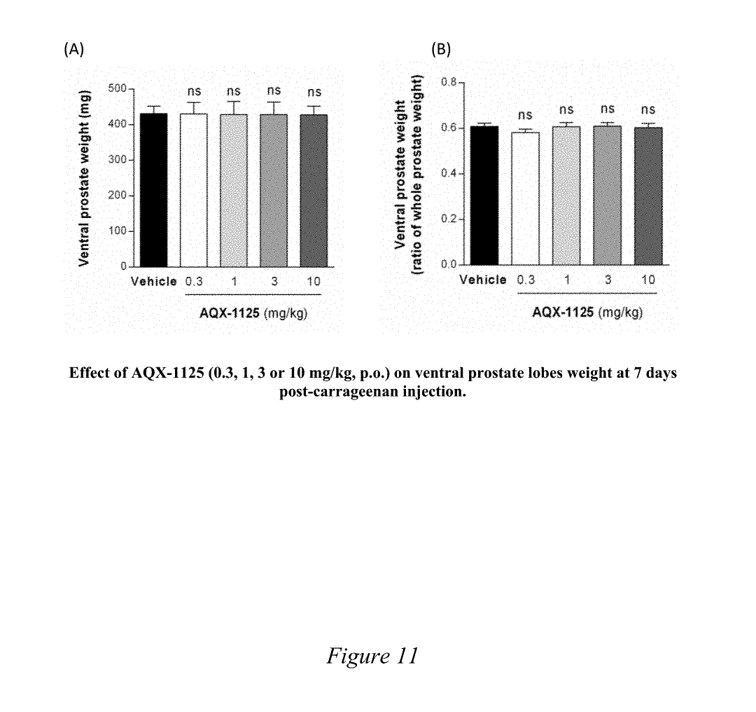

Seven days after carrageenan injection, AQX-1125 dosed at 0.3, 1, 3 or 10 mg/kg had no effect on ventral prostate lobes weights, expressed in mg or as a ratio of whole prostate weight (p<0.05, FIGS. 11A and 11B, respectively).

The aim of the above-described study was to determine the low end of the dosing range for AQX-1125 anti-nociceptive efficiency in the same model of inflammatory CP/CPPS induced by carrageenan in male Sprague-Dawley rats. AQX-1125 effects were evaluated on pelvic pain responses at 0.3, 1, 3 and 10 mg/kg doses (oral administration). The above results demonstrated that repeated oral treatments of AQX-1125 (once a day during 11 days at the three doses of 0.3, 1 and 3 mg/kg) had strong anti-nociceptive properties on carrageenan-induced pelvic pain. The maximal effect of AQX-1125 in the study was reached at a dose of 3 mg/kg.

The above-described study confirmed that a single i.prost. injection of carrageenan induced long lasting pelvic pain, including allodynia. No significant change of ventral prostate lobe weights was observed 7 days after carrageenan injection.

The above-described study demonstrated that, 1 h after its oral administration, ibuprofen (300 mg/kg) alleviated carrageenan-induced pelvic pain, thereby validating the model.

Daily oral treatments of AQX-1125, dosed at 0.3, 1, and 3 mg/kg, caused significant and dose-related decreases in carrageenan-induced pelvic pain. Beyond the optimal dose of 3 mg/kg, AQX-1125 had less pronounced anti-nociceptive properties.

It is noteworthy that AQX-1125 global effects were reproducible across the entire study period with similar results obtained at day 1, 3 and 7 post-carrageenan injection, and where pain evaluation was consistently performed 2 h post-treatment.

In conclusion, the above-described study demonstrated that the optimal dose of AQX-1125 is 3 mg/kg for pain management in a rat model of carrageenan-induced chronic prostatitis. Accordingly, AQX-1125 would be useful in treating prostatitis, particularly inflammatory CP/CPPS.

### Methods of Treatment and Administration

In order to treat a mammal having prostatitis, AQX-1125 may be formulated as a pharmaceutical composition in a manner similar to the pharmaceutical compositions disclosed in U.S. Pat. Nos. 7,601,874 and 7,999,010. Such pharmaceutical compositions comprise AQX-1125 and one or more pharmaceutically acceptable excipients, wherein AQX-1125 would be present in the composition in an amount that is therapeutically effective to treat prostatitis. Typically, the pharmaceutical compositions comprise AQX-1125 in an amount ranging from about 0.1 mg to about 300 mg per day, depending upon the route of administration, and more typically from about 1 mg to about 225 mg per day.

The pharmaceutical compositions of the invention can be administered to a mammal in need thereof once a day, twice a day, three times a day or four times a day. Preferably, the pharmaceutical compositions of the invention are administered to a mammal in need thereof once a day.

Appropriate concentrations and dosages can be readily determined by one skilled in the art. Depending upon the mammal, the therapeutically effective amount of AQX-1125 can be in the range of 0.1 mg/kg/day to 30 mg/kg/day.

Preferably, the pharmaceutical compositions of the invention comprise AQX-1125 in an amount that provides to a mammal in need thereof a daily dose of AQX-1125 from about 0.1 mg/kg to about 5.0 mg/kg; more preferably from about 0.2 mg/kg to about 4.0 mg/kg; even more preferably from about 0.3 mg/kg to about 3.75 mg/kg; and most preferably from about 2.0 mg/kg to about 3.5 mg/kg.

The pharmaceutically acceptable excipients present in the pharmaceutical compositions of the invention are familiar to those skilled in the art. For compositions formulated as liquid solutions, acceptable excipients include saline and sterile water, and may optionally include antioxidants, buffers, bacteriostats and other common additives. The compositions can also be formulated as pills, capsules, granules, or tablets which contain—in addition to AQX-1125—diluents, dispersing and surface-active agents, binders, lubricants, and/or delayed releases agents. One skilled in this art may further formulate AQX-1125 in an appropriate manner, and in accordance with accepted practices, such as those disclosed in Remington's Pharmaceutical Sciences, Gennaro, Ed., Mack Publishing Co., Easton, Pa. (current edition, the relevant sections of which are incorporated herein by reference in their entirety).

The methods of treating prostatitis as disclosed herein include administering to a mammal, preferably a human, AQX-1125 in an amount sufficient to treat prostatitis. Such methods include systemic administration of AQX-1125, preferably in the form of a pharmaceutical composition as discussed above. As used herein, systemic administration includes oral and parenteral methods of administration. For oral administration, suitable pharmaceutical compositions include powders, granules, pills, tablets, and capsules as well as liquids, syrups, suspensions, and emulsions. These compositions may also include flavorants, preservatives, suspending, thickening and emulsifying agents, and other pharmaceutically acceptable additives. For parenteral administration, the compounds of the present invention can be prepared in aqueous injection solutions which may contain buffers, antioxidants, bacteriostats, and other additives commonly employed in such solutions.

All of the U.S. patents, U.S. patent application publications, U.S. patent applications, foreign patents, foreign patent applications and non-patent publications referred to in this specification are incorporated herein by reference in their entireties.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. A method of treating prostatitis in a mammal, wherein the method comprises administering to the mammal in need thereof a therapeutically effective amount of AQX-1125.

2. The method of claim 1 wherein the prostatitis is selected from acute bacterial prostatitis, chronic bacterial prostatitis, chronic prostatitis/chronic pelvic pain syndrome (CP/CPPS) and asymptomatic inflammatory prostatitis.

3. The method of claim 1 wherein the prostatitis is acute bacterial prostatitis.

4. The method of claim 1 wherein the prostatitis is chronic bacterial prostatitis.

5. The method of claim 1 wherein the prostatitis is asymptomatic inflammatory prostatitis.

6. The method of claim 1 wherein the prostatitis is chronic prostatitis/chronic pelvic pain syndrome (CP/CPPS).

7. The method of claim 1 wherein the therapeutically effective amount of AQX-1125 administered to the mammal is from about 0.1 mg/kg to about 5.0 mg/kg.

8. (canceled)

9. The method of claim 7 wherein the therapeutically effective amount of AQX-1125 administered to the mammal is from about 0.2 mg/kg to about 4.0 mg/kg.

10. The method of claim 7 wherein the therapeutically effective amount of AQX-1125 administered to the mammal is from about 0.3 mg/kg to about 3.75 mg/kg.

11. The method of claim 7 wherein the therapeutically effective amount of AQX-1125 administered to the mammal is from about 2.0 mg/kg to about 3.5 mg/kg.

12. The method of claim 1 wherein the therapeutically effective amount of AQX-1125 is administered to the mammal once a day, twice a day, three times a day or four times a day.

13. The method of claim 12 wherein the therapeutically effective amount of AQX-1125 is administered to the mammal once a day.

14. The method of claim 1 wherein the therapeutically effective amount of AQX-1125 is orally administered to the mammal.

15. A method of treating prostatitis in a mammal, wherein the method comprises administering a therapeutically effective amount of a pharmaceutical composition comprising a pharmaceutically acceptable excipient and a therapeutically effective amount of AQX-1125 to the mammal in need thereof.
